(19) United States
(12) Patent Application Publication
(10) Pub. No.: US 2025/0102531 A1
(43) Pub. Date: Mar. 27, 2025

(54) COMPACT AUTOMATED BIOLOGICAL SEQUENCING DEVICE

(71) Applicant: MRIGlobal, Kansas City, MO (US)

(72) Inventors: Joseph A. Bogan, JR., Germantown, MD (US); Jennifer Stone, Melbourne, FL (US); Alec Jorns, Lake Quivira, KS (US); Joseph A. Russell, Silver Spring, MD (US)

(21) Appl. No.: 18/891,251

(22) Filed: Sep. 20, 2024

Related U.S. Application Data
(60) Provisional application No. 63/584,452, filed on Sep. 21, 2023.
(60) Provisional application No. 63/558,428, filed on Feb. 27, 2024.

(51) Int. Cl. G01N 35/00; C12Q 1/6869; G01N 35/10; G16B 35/20

(52) U.S. Cl. CPC G01N 35/00732; C12Q 1/6869; G01N 35/10; G16B 35/20; G01N 2035/00346

(57) ABSTRACT

A sequencing device as provided herein includes a cartridge configured to receive a sample of a biological material and prepare the sample for sequencing, a sequencer configured to sequence the prepared sample, and a container configured to receive the cartridge. The cartridge may include a nucleic acid extraction component, a mixing block, and a plurality of tubes for preparing the sample for sequencing. Two actuators may be provided within the container and may be configured to move the tubes to the sequencer via magnets. The container may be portable, such that sequencing may be completed using the sequencing device outside of a lab setting. The cartridge may be removable from the container and may be disposable and configured for one-time use. The operation of the device may be entirely automated.

### CROSS-REFERENCE TO RELATED APPLICATIONS

This application claims priority to U.S. Provisional Patent Application No. 63/584,452, filed Sep. 21, 2023, to Joseph Bogan et al., entitled “COMPACT BIOLOGICAL SEQUENCING DEVICE,” currently pending, and further claims priority to U.S. Provisional Patent Application No. 63/558,428, filed Feb. 27, 2024, to Joseph Bogan et al., entitled “COMPACT AUTOMATED BIOLOGICAL SEQUENCING DEVICE,” currently pending. The entire disclosures of the above references are incorporated herein by reference.

### FIELD OF THE INVENTION

The present invention relates to a biological sequencing device, and in particular a compact automated biological sequencing device which may be used for identification of biological materials in the field.

### BACKGROUND OF THE INVENTION

A standard biological sequencing device may be used to analyze and test samples of biological materials. Such devices may function to determine the order or sequence of the following bases from a nucleic acid preparation of DNA and/or RNA: A (adenine); C (cytosine); G (guanine); and T (thymine)/U (Uracil). The sequence order can be used to identify the biological material and/or components or characteristics of the biological material. Such devices include: a nucleic acid extraction component for extracting the nucleic acid of a biological test sample; a library preparation component for modification of the extracted nucleic acid in order to be processed by the sequencing device; and a sequencer component for sequencing and basecalling the sample and a bioinformatics component that analyzes and processes the sequencing results for interpretation.

Existing systems and devices are configured for lab settings and require large-scale equipment. Existing systems and devices also require a large amount of human intervention in order to complete a sequence of manual steps to modify the extracted nucleic acid for processing (e.g., moving the sample from step to step during the sequencing process). For example, in a lab setting, following nucleic acid extraction, a human must manually perform the library preparation process and manually transfer the library preparation into a sequencer component. Each of these may lead to human error and ultimately, inaccurate sequencing data. These systems additionally require large amounts of space for completing the entire process and substantial power requirements. Additionally, existing systems may include large equipment which may be difficult to transport and replace.

Accordingly, a need exists for a compact biological sequencing device. Additionally, a need exists for an automated biological sequencing device which does not require human intervention.

### SUMMARY OF THE INVENTION

The present invention generally relates to a compact automated biological sequencing device. The sequencing device may include: a container or housing; one or more cartridges that include a nucleic acid extraction component (referred to as the “extraction cartridges”); a library preparation component for preparing a sample for sequencing; a plurality of pumps, valves, actuators and/or magnets for operating the extraction cartridges and transporting or processing fluids; a sequencing cartridge; a sequencer for sequencing the processed test sample; and a computer system configured to process the sequence data for interpretation. The extraction cartridges may be configured to receive a sample, interface with a nucleic acid extraction system, prepare the extracted test sample for insertion into the sequencer for sequencing, and load the sequencer with the prepared sample. The extraction cartridges may be disposable and the preparation of a sample for sequencing may occur entirely within the cartridges such that there is no risk of contamination from previous samples and no requirement to thoroughly clean the device between each use. The extraction cartridges may include a plurality of channels and/or tubes that may be configured to transport a portion of the sample within the cartridge in order to prepare the sample for sequencing analysis within the device. The extraction cartridges, via the plurality of channels and/or tubes, may transport the sample to the sequencer.

The device may be portable and/or automated so that the sequencing may be completed outside of a lab setting (e.g., point of need and point of care settings). The device may be a fully self-contained device which may automate sample input, nucleic acid extraction, library preparation, sequencer loading, sequencing, and bioinformatics. Once a test sample is inserted into an extraction cartridge, the device completes all processing steps of the test sample and inserts the processed sample into the sequencer for analysis through an automated process. Upon completion, the extraction cartridge may be disposed, and new cartridges may be used to conduct testing of another sample. The extraction cartridges may be easily removable, and the processing tubes may or may not be permanently attached to any component of the device so that the extraction cartridge can be replaced without any user interaction to connect tubing to other components of the device. The device may include an on-board computing device and/or a display for processing, storing, and displaying information regarding tested samples. The device may also or alternatively be electronically and communicably coupled to an external computing device and/or display for processing, storing, and displaying information regarding tested samples. The device may also be configured with various integrated or wireless connection features (Bluetooth, Wi-Fi, etc.) that allow the device to be in communication with one or more external computing devices.

The present invention is further directed to a system for automatically sensing a biological material within the surrounding environment and analyzing a collected sample of the biological material to determine the identity or type of the biological material and/or one or more properties or characteristics of the biological material. The system may include a computer module, a trigger, a collector, and a biological material sequencing device. The computer module may be electronically and/or communicably coupled to the trigger, collector and sequencing device. The trigger may automatically detect the presence of a biological material within the air of the surrounding environment. Once detected, the computer module may direct the collector to collect a sample of the air with the biological material as a wet buffer sample. The computer module may then direct the collector to transfer the sample to the sequencing device to process the sample as described above.

Other aspects and advantages of the present invention will be apparent from the following detailed description of the preferred embodiments and the accompanying drawing figures.

### DESCRIPTION OF THE SEVERAL VIEWS OF THE DRAWINGS

The accompanying drawings form a part of the specification and are to be read in conjunction therewith, in which like reference numerals are employed to indicate like or similar parts in the various views.

FIG. 1 is a perspective view of a compact automated biological sequencing device in accordance with one embodiment of the present invention;

FIG. 2 is a perspective view of the device of FIG. 1 illustrating a cartridge used in the device in accordance with one embodiment of the present invention;

FIGS. 3A and 3B are perspective views of the cartridge used in the device of FIG. 1 and an actuator and sequencer used in the device in accordance with one embodiment of the present invention;

FIG. 4 is a perspective view of the device of FIG. 1 and illustrating the interior components of the device in accordance with one embodiment of the present invention;

FIG. 5 is a flowchart of a sequence achieved using the device of FIG. 1 in accordance with one embodiment of the present invention; and

FIG. 6 is a block diagram of a system with the compact automated biological sequencing device of FIG. 1 in accordance with one embodiment of the present invention.

### DETAILED DESCRIPTION OF THE INVENTION

The following detailed description of the invention references specific embodiments in which the invention can be practiced. The embodiments are intended to describe aspects of the invention in sufficient detail to enable those skilled in the art to practice the invention. Other embodiments can be utilized, and changes can be made without departing from the scope of the present invention. The present invention is defined by the appended claims and the description is, therefore, not to be taken in a limiting sense and shall not limit the scope of equivalents to which such claims are entitled.

The present invention is generally directed toward a compact automated biological sequencing device 10 as illustrated throughout the figures. The device 10 may be used to analyze biological materials by creating a nucleic acid preparation from a test sample of the material, preparing the sample for sequencing, and analyzing the sample in a sequencer-all in an automated manner. The device 10 may be configured to carry out four main functions: (1) nucleic acid extraction; (2) library preparation; (3) sequencing; and (4) bioinformatics. The device 10 may be used for any types of biological samples and organisms, including, but not limited to, bacterial, viral, fungal, parasite, prion, plant, animal, human, and/or other types of biological materials. In one particular application, the device 10 can be configured for analyzing engineered biological materials. According to one embodiment, the sequencing and testing completed on the device 10 is automated. As a result, the device 10 may reduce the potential for human error during the sequencing process and may be suitably used in the field where laboratory settings are not possible or practical.

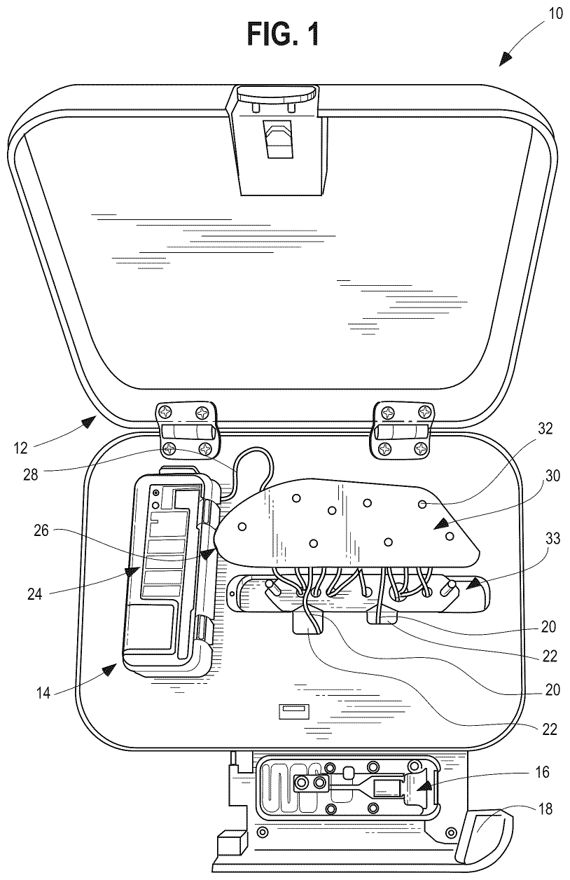

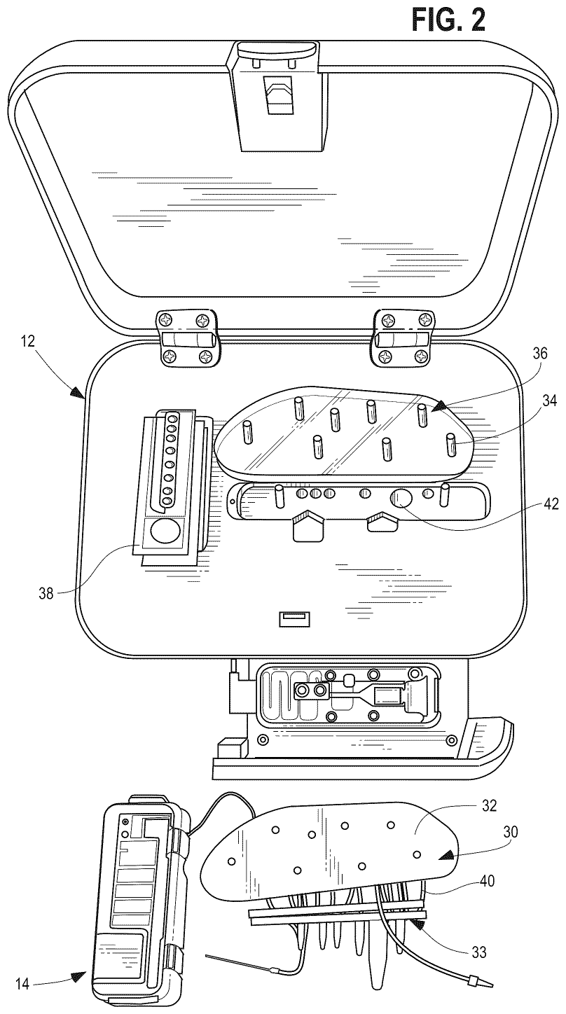

As best shown in FIGS. 1 and 2, the device 10 may include a container 12, which may house and/or receive the remaining components of the device 10. According to one embodiment, the container 12 may enable the device 10 to be portable by housing all components of the device 10 within a small defined footprint to facilitate transport and storage and allow the device 10 to be used in a variety of environments. The portability of the device 10 may solve the problems mentioned herein with respect to existing biological sequencing devices and systems. Currently known biological sequencing devices and systems require a traditional laboratory setting or portable laboratory workbench in order to accommodate the many components and processes required for biological sequencing of a specimen, some of which must be carried out manually. The device 10 is fully contained within the container 12 so that the device 10 may be transported and used in remote settings and may be used outside of a lab setting. The processes of the device 10 may automate all processing steps required for sequencing of the test sample within the container 12 so that laboratory settings (permanent or portable) are not necessary and precise human intervention is not needed to complete the sequencing. The device 10 may be powered by any power source known in the art, including a battery or socket-plug connection.

The device 10 may include a cartridge 14, which may be disposable and may be configured for one-time use. Such disposability may solve the problems mentioned herein with respect to existing biological sequencing devices and systems, including the requirement in existing devices to thoroughly clean any tubes or channels used in preparing a sample for sequencing to reduce the risk of sample to sample carry-over. In existing systems, typically, preparation of a particular biological fluid sample for sequencing requires the sample to be processed with one or more reagents prior to being sequenced. This processing in existing systems is typically completed manually by a user by processing the fluid sample and reagents through a plurality of tubes and pumps. In order to avoid potential contamination from prior tested fluid samples, the tubes are typically thoroughly cleaned between each use. The device 10 and the cartridge 14 of the present invention are configured to resolve these problems (including potential contamination from prior tested fluid samples) by making the cartridge 14 self-contained and disposable.

The cartridge 14 may be configured to process a biological fluid sample so that it may be sequenced and analyzed by a sequencer 16 as described in greater detail below. As best shown in FIGS. 1 and 2, the cartridge 14 may be inserted into the container 12 in order to be placed in fluid communication with the sequencer 16. After the cartridge 14 prepares the sample, the sample is inserted into the sequencer 16 to generate the A, C, G, T/U sequence of the sample. The sequencer 16 may be a flow cell sequencer where the prepared fluid sample, which may be processed by the cartridge 14 (via the container 12 and the components of the device 10 as described herein), is placed for sequencing. The sequencer 16 may be a sequencer known in the art, and in one non-limiting embodiment, the sequencer 16 may be a MinION® sequencer and the associated cartridge or “flow cell.” It is also recognized that any suitable sequencer device now known or hereinafter developed may be used as the sequencer 16. According to one embodiment, the device 10 includes an on-board computing device with a data library or information tool to analyze the output of the sequencer. According to one embodiment, a data library may be uploaded to the device 10 such that the sequencer 16 may not need to be connected to a separate computing device and/or may not need internet connectivity to complete testing and analysis of the sample in the device 10. In addition, or alternatively, the device 10 may be configured with standard wired or wireless internet connection technologies to allow for communication with remote networks containing data libraries for the sequencer 16. The device 10 may also include and/or be electronically and communicably coupled with an external computing device configured to operate the device 10 and contain a data library or information tool to analyze the output of the sequencer. The sequencer 16 may be insertable into the container 12 so that it may be in fluid communication with the cartridge 14. According to one embodiment as shown in FIGS. 1 and 2, the sequencer 16 may be placed on a removable drawer 18 of the container 12, such that when the drawer 18 is fully received within the container 12, the sequencer 16 may be hidden and may be protected from the elements.

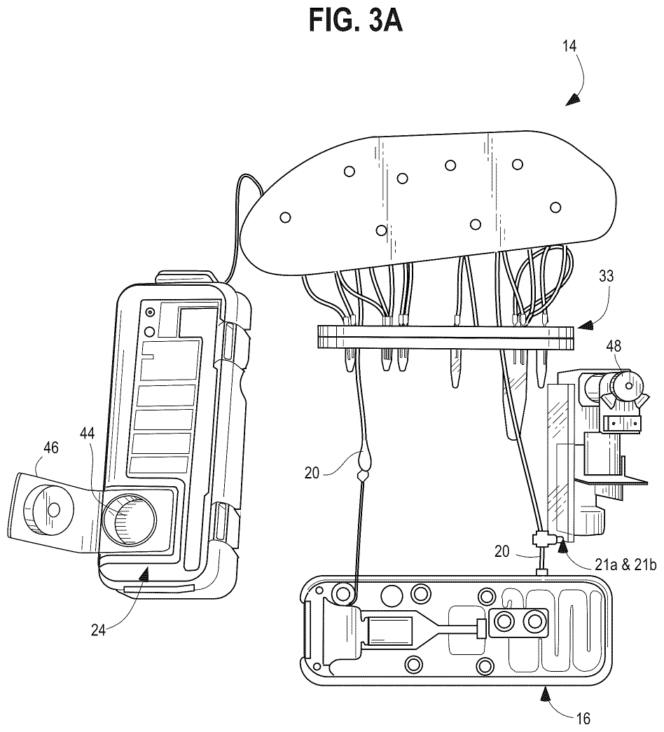

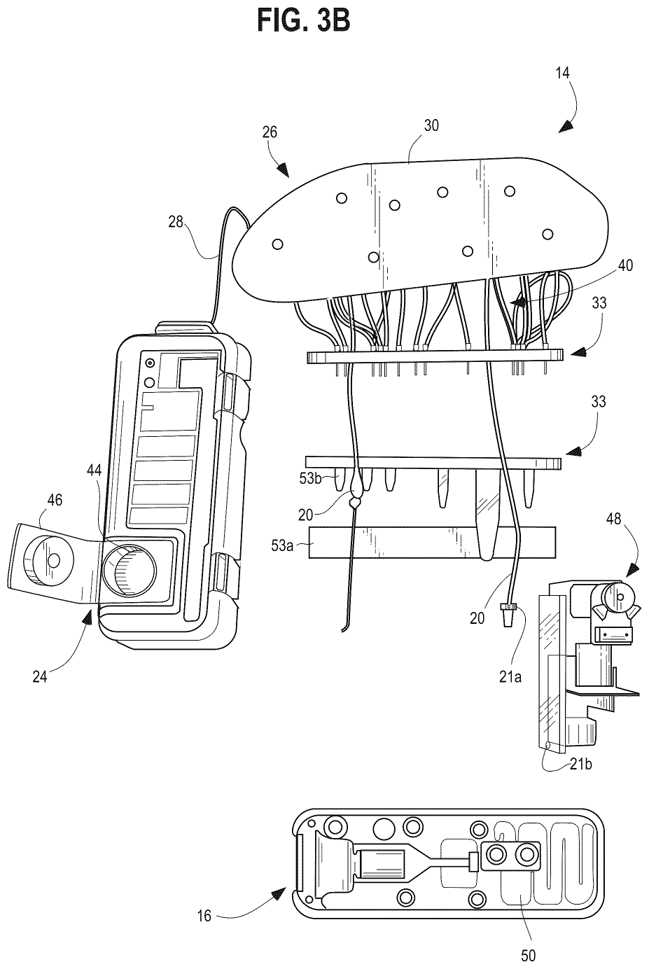

The cartridge 14 may interact with and fluidly connect to the sequencer 16 via a plurality of sequencing tubes 20. As best illustrated in FIGS. 3A and 3B, the cartridge 14 may include two sequencing tubes 20 that are configured to extend toward the sequencer 16 in order to deposit the prepared fluid sample into the sequencer 16; however, there may be any number of sequencing tubes 20 depending on the desired configuration and particular type of the sequencer 16. According to one embodiment, the sequencing tubes 20 are inserted and received through a plurality of cutouts 22 of the container 12 (see FIGS. 1 and 2) so that they may extend into the interior of the container 12 where the sequencer 16 is located after being inserted into the container 12 through the drawer 18. The number of sequencing tubes 20 may correspond to the number of the plurality of cutouts 22. For example, if there are two sequencing tubes 20, there may be two cutouts 22.

As best shown in FIGS. 3A and 3B, the sequencing tubes 20 may include a magnetic attachment block 21a provided at the lower end of each of the sequencing tubes 20 to facilitate alignment of the sequencing tubes 20 with a plurality of receiving ports 52 on the sequencer 16. The number of sequencing tubes 20 may correspond to the number of the plurality of receiving ports 52. For example, if there are two sequencing tubes 20, there may be two receiving ports 52. As described in greater detail herein, the device 10 may include one or more actuators 48 that may each have a corresponding magnetic attachment block 21b so that, when the sequencing tubes 20 are located within the interior of the container 12 via the cutouts 22, the sequencing tubes 20 can attach to the one or more actuators 48. FIG. 3A illustrates one of the sequencing tubes 20 attached to one of the one or more actuators 48 via the magnetic attachment blocks 21a and 21b, and FIG. 3B illustrates one of the sequencing tubes 20 separated from one of the one or more actuators 48. The one or more actuators 48 may be configured to move the sequencing tubes 20 at the appropriate time and align the sequencing tubes 20 with the receiving ports on the sequencer 16 as described in greater detail below. According to one embodiment, the one or more actuators 48 may be configured as two-axis actuators. This may enable each of the one or more actuators 48 to move in two directions within the interior of the container 12 (i.e., x and z directions) so that the sequencing tubes 20 may align with the plurality of receiving ports 52 located at different points on the sequencer 16. Accordingly, each sequencing tube 20 may be moved by the actuators 48 in a first, or x, direction, and moved in a second, or y, direction to achieve a desired position of the sequencing tube 20. The plurality of receiving ports 52 on the sequencer 16 may be located at different axes, such that the sequencing tubes 20 may need to be aligned at different axes. The sequencing tubes 20 may include plugs which may be used to close receiving ports when not in use.

The cartridge 14 may include a sample receiving block 24 which may include reservoirs configured to receive a sample and extract and store nucleic acid components of the sample. The sample receiving block 24 may be configured as a nucleic acid extraction device that extracts nucleic acid from the received test sample and places the extracted nucleic acid into a collection reservoir. According to one embodiment, the sample receiving block 24 may be configured as the Biomeme® Integrated Sample Prep (ISP) device and test cartridge or other now known or hereinafter developed nucleic acid preparation component. The cartridge 14 may further include a fluidics cartridge portion 26 for receiving the sample component from the sample receiving block 24. The sample receiving block 24 may be fluidly connected to the fluidics cartridge portion 26 such that extracted nucleic acid sample from the sample receiving block 24 may be transported to the fluidics cartridge portion 26 via a transportation tube 28. According to one embodiment as best shown in FIGS. 2 and 3A-B, the sample receiving block 24 may be a stand-alone component (such as the Biomeme® Integrated Sample Prep (ISP) device) that is connected to the fluidics cartridge portion 26 and the remainder of cartridge 14. In certain alternative embodiments (not shown), the sample receiving block 24 may be incorporated into the fluidics cartridge portion 26 to provide a single, integrated cartridge component that both carries out the nucleic acid extraction process of the sample receiving block 24 and library preparation process of the fluidics cartridge portion 26 (as described in greater detail below) within a single component.

The fluidics cartridge portion 26 may include a base block 30, which may include a plurality of openings or holes 32. The plurality of openings 32 may align with a plurality of pump heads 34 of a pump head block 36, as shown in the disassembled view of the device 10 in FIG. 2. The plurality of pump heads 34 extend outward from the pump head block 36, which contains the corresponding pumps within the interior of the container 12. The plurality of pump heads 34 are configured to operate the cartridge 14 and move the fluid sample portions through a plurality of mixing tubes 40 for preparation of the sample for sequencing. Each of the plurality of openings 32 corresponds to one or more of the plurality of mixing tubes 40, which is wrapped around each of the plurality of openings 32 on the interior of the base block 30. Once the base block 30 is inserted onto the pump head block 36, the corresponding pump head of the plurality of pump heads 34 may operate to force the flow of liquid within one of the plurality of mixing tubes 40 that corresponds to each of the plurality of openings 32.

As further shown in FIG. 2, the fluidics cartridge portion 26 may include a mixing block 33, which houses a portion of the plurality of mixing tubes 40 and reservoirs containing reagents that are used for preparing the sample for sequencing. The mixing block 33 may be received within one or more corresponding slots or cutouts 42 defined into the container 12. The mixing block 33 may operate in a series of process steps for preparing the sample for sequencing as described in greater detail below.

As illustrated in FIGS. 1 and 2, the cartridge 14 may be inserted into and removable from the container 12. As shown, the sample receiving block 24 portion of the cartridge 14 may be inserted into a receptacle portion 38 defined into the container 12. The fluidics cartridge portion 26 may also be removable from the container 12. The base block 30 may be inserted onto the pump head block 36 so that each of the plurality of openings 32 is positioned onto a corresponding pump head of the plurality of pump heads 34. The mixing block 33 may also be inserted into a cutout of the cutouts 42 that is adjacent to the base block 30 and the pump head block 36. Once the base block 30 and the mixing block 33 are positioned onto and connected to the pump head block 36 and the cutouts 42, respectively, then the sequencing tubes 20 that extend from the base block 30 may be received within the cutouts 22 and into the interior of the container 12. After the cartridge 14 is fully connected to the container 12 and the remainder of the device 10, then the device 10 may operate to prepare and sequence a test sample.

Turning now to FIGS. 3A and 3B, the cartridge 14 may receive the sample at a sample receiving portion 44 on the sample receiving block 24. After the sample is received, a latch 46 may be lowered to cover the sample receiving portion 44 and seal the sample within the cartridge 14. Within the sample receiving block 24, the inserted sample may be processed into an extracted nucleic acid fluid sample, which is then transferred to the fluidics cartridge portion 26 by the transportation tube 28. Once transferred to the fluidics cartridge portion 26, the sample may be further prepared through a series of preparation processing steps via the plurality of pump heads 34, the plurality of mixing tubes 40, and the mixing block 33. The plurality of pump heads 34 are operated in a specific sequence to move the fluid sample from the sample receiving block 24 to the mixing block 33, where it is mixed in a specific sequence with one or more reagents contained within the reservoirs of the mixing block 33, and then finally to the sequencing tubes 20 as described in greater detail below. As further shown in FIG. 3B, the device 10 may include a heating block element 53a that may be used during the processing of the fluid sample within cartridge 14. According to one embodiment, a portion of the fluid sample may be heated by a heating block 53a within a heating tube 53b located on mixing block 33 as described in greater detail below. The heating block element 53a may be positioned within and affixed within container 12 so that the lower end of mixing block 33, including heating tube 53b is at least partially received within heating block element 53a once cartridge 14 is inserted into container 12.

As further illustrated in FIGS. 3A and 3B, once the prepared fluid samples are located in the sequencing tubes 20, the one or more actuators 48 may operate to move to the sequencing tubes 20 by attaching to the sequencing tubes 20 via the magnetic attachment blocks 21a and 21b. When one of the one or more actuators 48 is positioned in a location near one of the sequencing tubes 20, the magnetic attractive force between the magnetic attachment blocks 21a and 21b allow one of the sequencing tubes 20 to be attached to one of the one or more actuators 48 even if the two components are not fully aligned. Then the one or more actuators 48 may move the sequencing tubes to the sequencer 16, which is located in the interior of the container 12.

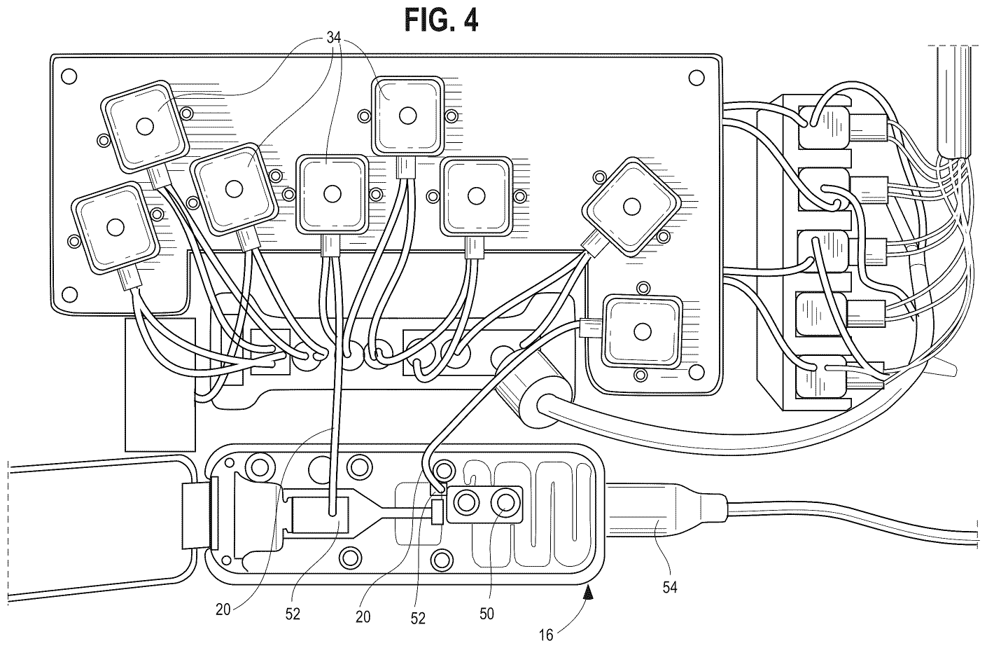

As illustrated in FIG. 4, the sequencer 16 may include two receiving ports 52 defined into the upper surface of the sequencer 16. The plurality of receiving ports 52 may be configured as a priming port and a spot-on port. The plurality of receiving ports 52 may be configured to receive the prepared sample solution from the sequencing tubes 20 of the cartridge 14. Each of the one or more actuators 48 may be configured to move one of the sequencing tubes 20 over one of the plurality of receiving ports 52 so that the prepared sample may be released into both of the plurality of receiving ports 52 for analysis and sequencing by the sequencer 16. As further shown in FIG. 4, the sequencer 16 may include a cover 50 which may enclose one of the plurality of receiving ports 52. The cover 50 may be opened when the prepared sample is inserted into one of the plurality of receiving ports 52, and the cover 50 may be closed when processing of the sample is being completed by the sequencer 16. According to one embodiment, the device 10 includes a servo motor (not shown) located in the interior of the container 12 with an arm (not shown) configured to open and close the cover 50 during operation of the device 10.

As further shown in FIG. 4, the sequencer 16 may include a connection 54 that connects the sequencer 16 to an internal computer (not shown) in the container 12. The computer can be configured to contain a data library and analysis tool (such as PanGIA as described below) to process the results and/or output from the sequencer 16 and then display the results on a display screen (not shown) located on the container 12. In alternative embodiments, the computer may be configured to send the results to an external or remote device for processing and/or display. In other alternative embodiments, the computer may be located external to container 12 and electronically and communicably coupled to the sequencer 16 and/or remainder of the device 10.

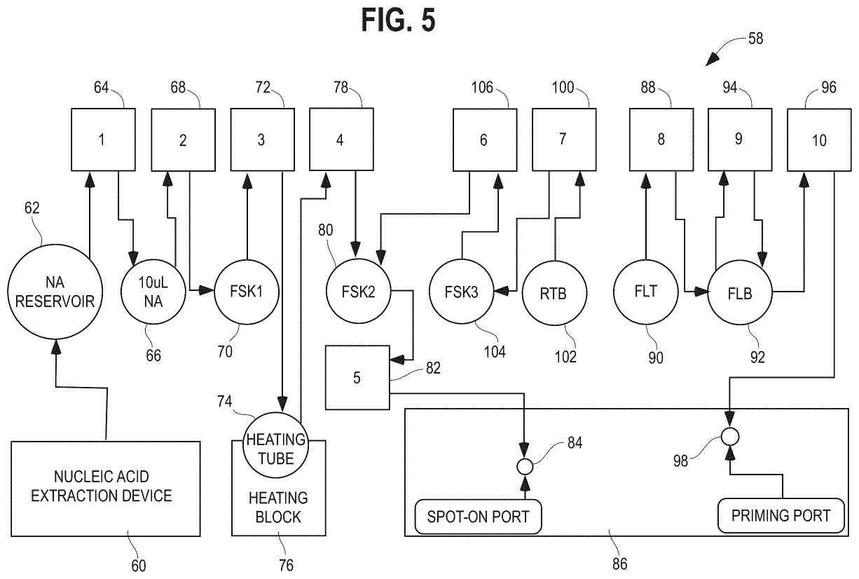

FIG. 5 provides a schematic representation of the device 10, according to one embodiment, and illustrates the sequencing and operating processes 58 carried out by the device 10 when analyzing a sample. As shown, the device 10 may include a nucleic extraction device 60 (which may be configured as the sample receiving block 24 described above) that may be configured to extract nucleic acids from a sample input (which may be a solution). The nucleic extraction device 60 may output a nucleic acid extract into a nucleic acid collection reservoir 62.

The plurality of pump heads 34 (as described above and positioned onto the pump head block 36) may transfer the solution for preparation. The plurality of pump heads 34 may include a forward and reverse drive peristatic pump. The plurality of pump heads 34 may transfer the solution through a plurality of flow channels (as illustrated by line arrows). The flow channels may schematically represent the fluidic movement of the sample through the plurality of mixing tubes 40 described above and included in the cartridge 14 between the base block 30 and the mixing block 33. The plurality of pump heads 34 may include a first pump 64 which may transfer the solution from the nucleic acid collection reservoir 62 to a nucleic acid tube 66. According to various embodiments, the solution may comprise a range of about 0 microliters (μL) to 1 mL of nucleic acid, a range of about to 5 μL to 75 μL of nucleic acid, or a range of about to 10 μL to 50 μL of nucleic acid. According to one embodiment, the solution may comprise 10 μL of nucleic acid.

The nucleic acid tube 66 may be a tube capable of transporting 10 μL of nucleic acid. A second pump 68 may transfer the solution from the nucleic acid tube 66 to a first fragmentation mixing (FSK) tube 70. The solution may be mixed with a lyophilized fragmentation mix that may found in the first FSK tube 70. A third pump 72 may transfer the solution from the first FSK tube 70 to a heating tube 74. The heating tube 74 may be heated by a heating block 76; the heating tube 74 and the heating block 76 may be the same as and/or similar to the heating tube 53b and heating block 53a, respectively, as discussed above with reference to FIG. 3B. The solution and lyophilized fragmentation mix may be incubated in the heating tube 74. In one embodiment, such incubation occurs for 1 minute. A fourth pump 78 may transfer the solution from the heating tube 74 to a lyophilized fragmentation mix rapid adapter or to a second FSK tube 80. A fifth pump 82 may mix the solution and the lyophilized fragmentation mix in the second FSK tube 80, which may produce a mixture of the solution and fragmentation mix. The second FSK tube 80 may be configured as one of the sequencing tubes 20 described above. The mixture may be incubated in the second FSK tube 80. According to one embodiment, the mixture is incubated in the second FSK tube 80 at room temperature (e.g., about 25° C.). According to one embodiment, the mixture is incubated in the second FSK tube 80 for about 5 minutes. The fifth pump 82 may transfer the mixture (from the second FSK tube 80/one of the sequencing tubes 20) to a spot-on port 84 of a flow cell 86. The spot-on port 84 may be configured as one of the plurality of receiving ports 52, and the flow cell 86 may be configured as the sequencer 16 as described above. According to one embodiment, the flow cell 86 comprises a MinION® flow cell sequencer device.

A sixth pump 88 may transfer a FSK flush tether reagent, which may be stored in a flush tube 90, to a flush buffer tube 92. This transfer may mix the FSK flush tether reagent with a FSK flush buffer reagent. A seventh pump 94 may mix the FSK flush tether reagent and the FSK flush buffer reagent in the flush buffer tube 92, which may be configured as one of the sequencing tubes 20 described above. An eighth pump 96 may transfer the mixture of the FSK flush tether reagent and the FSK flush buffer reagent to a priming port 98 of the flow cell 86. The priming port 98 may be one of the plurality of receiving ports 52, and the flow cell 86 may be the sequencer 16 in the device 10 as described above. According to one embodiment, the eighth pump 96 transfers about 800 μL of the mixture of the FSK flush tether reagent and the FSK flush buffer reagent to the priming port 98. The mixture may be incubated in the flow cell 86 for about 5 minutes.

A ninth pump 100 may transfer a FSK resuspension buffer, which may be stored in a FSK resuspension buffer tube 102, to a lyophilized FSK sequencing buffer, which may be stored in a third FSK tube 104. A tenth pump 106 may mix the FSK resuspension buffer and the FSK sequencing buffer in the third FSK tube 104. Before the FSK flush tether reagent and the FSK flush buffer reagent mixture is transferred to the priming port 98, the tenth pump 106 may transfer a FSK resuspension buffer and the FSK sequencing buffer mixture to the third FSK tube 104.

The fifth pump 82 may mix the solution and lyophilized fragmentation mixture with the FSK resuspension buffer and FSK sequencing buffer mixture. This mixture may be incubated in the second FSK tube 80. According to one embodiment, the mixture is incubated in the second FSK tube 80 for about 5 minutes. After the eighth pump 96 has transferred approximately 800 μL of the mixture of the FSK flush tether reagent and the FSK flush buffer reagent to the priming port 98 and incubated for 5 minutes, the eighth pump 96 may transfer an additional 200 μL of the FSK flush tether reagent and FSK flush buffer reagent mixture to the priming port 98. In one embodiment, immediately thereafter, the fifth pump 82 may transfer dropwise the contents of the second FSK tube 80 into the spot-on port 84 of the sequencer 16 in order to load the prepared sample into the sequencer 16.

The flow cell 86/sequencer 16 may initiate sequencing of the mixture of the FSK flush tether reagent and FSK flush buffer reagent and the mixture of the solution and lyophilized fragmentation mixture and the FSK resuspension buffer and FSK sequencing buffer mixture. Such sequencing may be processed by a bioinformatics analysis tool located on a computer provided within the device 10. Such bioinformatics analysis tool may be a PanGIA (Pan-Genomics for Infectious Agents) tool and/or any other analysis platform. The computer may be configured to identify the biological material of the sample and/or one or more components or characteristics of the biological material. The identity, components, characteristics, and/or sequencing of the material may be displayed on a display readout connected to the computer and included in the device 10. According to one embodiment, the sequencing may be associated with a certain confidence score, such that a user may determine how accurate a certain test or sequence may be.

The foregoing sequence steps and procedures describe a method of operating the device 10 in order to analyze and test a sample of a biological material. As described, the method includes the steps of (i) receiving a test sample of a biological material, (ii) extracting the nucleic acid components of the sample, (iii) preparing the sample for sequencing by mixing the nucleic acid extraction with one or more reagents and buffer solutions, heating the mixtures and/or incubating the mixtures, (iv) introducing prepared sample solutions to a sequencer, (v) sequencing the prepared solutions, (vi) sending the output to a computing device having a bioinformatics analysis tool, and (vii) analyzing the output via the analysis tool and outputting the analyzed results on a display. Each of the foregoing method steps may involve one or more sub-steps as described in greater details above with respect to FIG. 5.

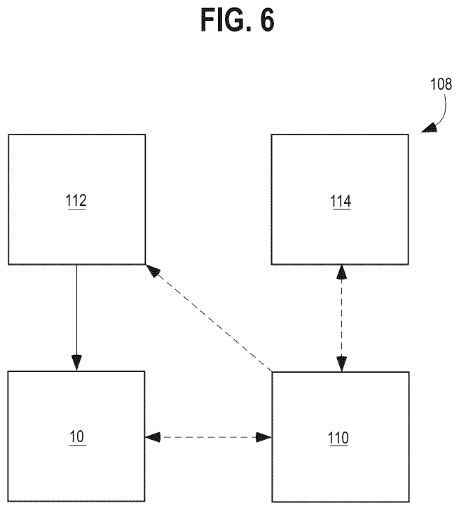

The present invention is further directed to a system 108 that incorporates the device 10 along with other components as schematically illustrated in FIG. 6. System 108 may be configured to automatically detect and analyze a biological material in a surrounding environment. System 108 may include a computer module 110, a collector 112, a trigger 114 and the device 10. According to one embodiment, the computer module 110 may be in electronic communication with the collector 112, the trigger 114 and the device 10. As illustrated in FIG. 6, dashed lines indicate electronic communication paths between each of the components. The computer module 110 may be a computer module known in the art, and in one embodiment, the computer module 110 is an Intel NUC computer module. It is also recognized that any suitable computer module now known or hereinafter developed may be used as the computer module 110. The collector 112 may be a collector known in the art, and in one embodiment, the collector 112 is a Coriolis Micro collector. It is also recognized that any suitable collector now known or hereinafter developed may be used as the collector 112. The trigger 114 may be a trigger known in the art, and in one embodiment, the trigger 114 is a S3i APC trigger. It is also recognized that any suitable trigger now known or hereinafter developed may be used as the trigger 114.

In various embodiments, the trigger 114 may be configured to automatically determine whether there is biological material in the air of an environment around the system 108. In one embodiment, the trigger 114 relies upon its internal technology and algorithms to determine whether there is biological material in the air. If the trigger 114 determines that there is biological material in the air, the trigger 114 may communicate with the computer module 110 based on this determination, which may cause the computer module 110 to communicate with the collector 112. The computer module 110 may prompt the collector 112 to take action; in one embodiment, the collector 112 may collect a sample of the air containing the biological material and may transform this sample into a wet buffer sample. The collector 112 may then pump the wet buffer sample into the device 10 for extraction, as described above. According to one embodiment, the collector 112 may include a conical tube which may feed the wet buffer sample into the device 10. As illustrated in FIG. 6, solid lines indicate fluid paths.

The device 10 may be in electronic communication with the computer module 110. As discussed above with reference to FIG. 5, sequencing that occurs in the device 10 may be processed by a bioinformatics analysis tool located on the computer module 110. Such bioinformatics analysis tool may be a PanGIA (Pan-Genomics for Infectious Agents) tool and/or any other analysis platform. The computer module 110 may be configured to identify the biological material of the sample and/or one or more components or characteristics of the biological material. The identity, components, characteristics, and/or sequencing of the material may be displayed on a display readout connected to the computer module 110.

From the foregoing, it will be seen that this invention is one well adapted to attain all the ends and objects hereinabove set forth together with other advantages which are obvious, and which are inherent to the structure. It will be understood that certain features and sub combinations are of utility and can be employed without reference to other features and sub combinations. This is contemplated by and is within the scope of the claims. Since many possible embodiments of the invention can be made without departing from the scope thereof, it is also to be understood that all matters herein set forth or shown in the accompanying drawings are to be interpreted as illustrative and not limiting. As one example, the description above and figures attached hereto illustrate automation of a DNA-only library prep chemistry (e.g., the Oxford Nanopore Field Sequencing Kit (FSK)). Other possible embodiments may automate different library preparation methods adapted to DNA and/or RNA sequencing as well as library preparation methods that may include nucleic acid amplification.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including”, and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

We claim:

1. A sequencing device for analyzing a biological material, the device comprising:
a cartridge configured to receive a sample of the biological material and prepare a sample preparation;
a plurality of pumps configured to operate the cartridge;
a sequencer configured to sequence the sample preparation and provide one or more outputs relating to the sample preparation;
a container configured to house the cartridge, the plurality of pumps, and the sequencer; and
a computer configured to identify one or more components or characteristics of the biological material based on the one or more outputs from the sequencer;
wherein:
the cartridge is removable from container;
the sample preparation is prepared entirely within the cartridge; and
the cartridge, the plurality of pumps, and the sequencer are located entirely within or on the container.

2. The device of claim 1, wherein the cartridge is disposable and is configured for one-time use.

3. The device of claim 1, wherein the cartridge comprises two sequencing tubes for transferring the sample preparation to the sequencer.

4. The device of claim 3, wherein the device further comprises two actuators located within the container, wherein the two actuators are two-axis actuators.

5. The device of claim 4, wherein the two sequencing tubes each include a first magnet attachment block, and wherein the two actuators each include a second magnet attachment block, and wherein the actuators are configured to attach to the sequencing tubes through the first and second magnet attachment blocks in order to place the sequencing tubes above the sequencer.

6. The device of claim 1, wherein the cartridge comprises:
a sample receiving block configured to receive the sample of the biological material and extract nucleic acid components from the sample;
a base block having a plurality of tubes extending therefrom and a plurality of openings mechanically connected to the plurality of tubes, wherein each opening of the plurality of openings is configured to receive one pump of the plurality of pumps;
a mixing block in fluid communication with the base block and the plurality of tubes, the mixing block configured to process the nucleic acid components of the sample with one or more reagents to form the sample preparation; and
at least one sequencing tube in fluid communication with the base block and the plurality of tubes, the at least one sequencing tube configured to transfer the sample preparation to the sequencing device.

7. The device of claim 6, wherein the preparation of the sample and the sequencing of the sample preparation is automated within the device after the sample is received within the sample receiving block.

8. The device of claim 1, wherein the computer includes at least one of a library database and a bioinformatics tool to determining the one or more components or characteristics of the biological material.

9. The device of claim 1, wherein the container comprises a drawer for inserting and removing the sequencer within an interior of the container.

10. A method of operating the device of claim 1, comprising the steps of:
receiving the sample of the biological material;
extracting the nucleic acid components of the sample;
preparing the sample for sequencing to form the sample preparation by performing at least one of mixing the nucleic acid extraction with one or more reagents and buffer solutions, heating the mixtures, and incubating the mixtures;
inserting the sample preparation into the sequencer;
sequencing the sample preparation; and
sending the output to the computer and analyzing the output via the computer to identify one or more components or characteristics of the biological material.

11. A system comprising:
a computer module;
a trigger, wherein the trigger is configured to determine whether there is a biological material in air around the trigger;
a collector, wherein the collector is configured to transform the biological material in the air into a sample of the biological material; and
a sequencing device for analyzing the biological material, the device comprising:
a cartridge configured to receive the sample of the biological material and prepare a sample preparation;
a plurality of pumps configured to operate the cartridge;
a sequencer configured to sequence the sample preparation and provide one or more outputs relating to the sample preparation; and
a container configured to house the cartridge, the plurality of pumps, and the sequencer.

12. The system of claim 11, wherein the computer module is configured to identify one or more components or characteristics of the biological material based on the one or more outputs from the sequencer.

13. The system of claim 11, wherein the trigger, the collector, and the sequencing device are in electronic communication with the computer module.

14. The system of claim 11, wherein:
the cartridge is removable from container;
the sample preparation is prepared entirely within the cartridge; and
the cartridge, the plurality of pumps, and the sequencer are located entirely within or on the container.

15. The system of claim 11, wherein the cartridge is disposable and is configured for one-time use.

16. The system of claim 11, wherein the cartridge comprises two sequencing tubes for transferring the sample preparation to the sequencer.

17. The system of claim 16, wherein the device further comprises two actuators located within the container, wherein the two actuators are two-axis actuators.

18. The system of claim 17, wherein the two sequencing tubes each include a first magnet attachment block, and wherein the two actuators each include a second magnet attachment block, and wherein the actuators are configured to attach to the sequencing tubes through the first and second magnet attachment blocks in order to place the sequencing tubes above the sequencer.

19. The system of claim 11, wherein the cartridge comprises:
a sample receiving block configured to receive the sample of the biological material and extract nucleic acid components from the sample;
a base block having a plurality of tubes extending therefrom and a plurality of openings mechanically connected to the plurality of tubes, wherein each opening of the plurality of openings is configured to receive one pump of the plurality of pumps;
a mixing block in fluid communication with the base block and the plurality of tubes, the mixing block configured to process the nucleic acid components of the sample with one or more reagents to form the sample preparation; and
at least one sequencing tube in fluid communication with the base block and the plurality of tubes, the at least one sequencing tube configured to transfer the sample preparation to the sequencing device.

20. The system of claim 11, wherein the computer module includes at least one of a library database and a bioinformatics tool to determining the one or more components or characteristics of the biological material.
